(19) United States
(12) Patent Application Publication
(10) Pub. No.: US 2022/0265585 A1
(43) Pub. Date: Aug. 25, 2022

(54) Methods and Compositions to Inhibit Symptoms Associated with Opioid Withdrawal

(71) Applicant: SEN-JAM PHARMACEUTICAL LLC, Huntington, NY (US)

(72) Inventors: Jacqueline Iversen, Lloyd Harbor, NY (US); Thomas A. Dahl, Guilford, CT (US)

(21) Appl. No.: 17/690,680

(22) Filed: Mar. 9, 2022

Related U.S. Application Data
(63) Continuation of application No. 16/133,398, filed on Sep. 17, 2018, now abandoned.
(60) Provisional application No. 62/560,023, filed on Sep. 18, 2017.

(51) Int. Cl. A61K 31/192; A61K 9/68; A61P 25/36; A61K 9/00

(52) U.S. Cl. CPC A61K 31/192; A61K 9/0058; A61P 25/36; A61K 9/0056; A61K 31/4535

(57) ABSTRACT

The present invention provides a method of inhibiting symptoms associated with opioid withdrawal in a human subject in need thereof. The method comprises administering an effective amount of a pharmaceutical composition to the subject during an opioid withdrawal period, wherein the pharmaceutical composition comprises: a) a non-steroidal anti-inflammatory drug (NSAID); and b) a co-agent selected from the group consisting of: fexofenadine, ketotifen, desloratadine and cetirizine and combinations thereof.

### FIELD OF THE INVENTION

The present invention relates to methods, and compositions, for inhibiting adverse symptoms associated with opioid withdrawal.

### BACKGROUND OF THE INVENTION

Opioids are a class of drugs that include the illegal drug heroin and pain relievers available legally by prescription, such as oxycodone (OxyContin®), hydrocodone (Vicodin®), codeine and morphine. Prescription opioid pain relievers are generally safe when taken for a short time and as prescribed by a doctor. However, due to the euphoria induced by opioids, they are frequently misused. In fact, the misuse of and addiction to opioid drugs can be initiated after exposure to prescribed medications in a clinic or physician's office. As many as 90% of patients in chronic pain management settings receive opioid pain relievers, and the prevalence of drug abuse is 9-41% among these patients (Manchikanti, L., Pain Physician, 2006. 9(4): 287-321). Moreover, even as prescribed by a doctor, regular use of opioids can lead to dependence.

Addiction to illicit and prescription opioids is a significant public health issue. Each month in the United States, 4.9% of persons aged 12 or older (>11 million) use prescription pain relievers for non-medical purposes. Young adults (age 18 to 25) are particularly hard hit by this problem, and they have the highest rate of abuse of prescription pain relievers (Results from the 2006 National Survey on Drug Use and Health: National Findings. Substance Abuse and Mental Health Services Administration, Department of Health and Human Services, 2006). In 2013, between 28 and 38 million people used opioids illicitly (0.6% to 0.8% of the global population between the ages of 15 and 65) (“Status and Trend Analysis of Illicit Drug Markets,” World Drug Report 2015). In 2011, an estimated 4 million people in the United States used opioids recreationally or were dependent on them. Current increased rates of recreational use and addiction are attributed to over-prescription of opioid medications and inexpensive illicit heroin.

Opioid addiction has devastating consequences for personal health and society (Birnbaum et al., Clin J Pain, 2006. 22(8):667-676; Gruber et al. Neuropsychol Rev. 2007. 17(3):299-315). Drug overdose is the leading cause of injury death in the United States. The American Society of Addiction Medicine (ASAM) estimates that 100 Americans die of a drug overdose every day, and 46 people in the United States die daily due to prescription opioid overdose. The New York Times reports that opioids are responsible for more deaths than any other medication or drug (NYT Dec. 22, 2014).

Withdrawal from opioid use is difficult. An addicted, or depended, individual experiences adverse symptoms including anxiety, increased pain sensitivity, poor concentration, tachycardia and flu-like symptoms during withdrawal, a syndrome reflecting physical dependence on these drugs (Handelsman et al., Am J Drug Alcohol Abuse, 1987. 13(3):293-308). Such adverse withdrawal symptoms are a major contributor to the addictive nature of opioids. Current strategies for treatment of opioid withdrawal are suboptimal; they rely on the administration of controlled substances (methadone and buprenorphine), or medications with significant hemodynamic side effects (clonidine).

Due to the potential dire consequences of opioid use, there is an urgent need to assist individuals to withdraw from such use. Adverse symptoms experienced during the opioid withdrawal period are a deterrent to ending opioid use, especially an opioid addiction. Accordingly, there is an urgent need to inhibit such symptoms to enable a successful opioid withdrawal.

### SUMMARY OF THE INVENTION

In one embodiment, the present invention is a method of inhibiting symptoms associated with opioid withdrawal in a human subject in need thereof. The method comprises administering an effective amount of a pharmaceutical composition to the subject during an opioid withdrawal period. The pharmaceutical composition comprises a) a non-steroidal anti-inflammatory drug (NSAID); and b) a co-agent selected from the group consisting of: fexofenadine, ketotifen, desloratadine and cetirizine and combinations thereof. The symptoms associated with opioid withdrawal are inhibited in the human subject.

Examples of the NSAID include aspirin, ibuprofen, naproxen, diclofenac, diflunisal, etodolac, indomethacin, ketoprofen, ketorolac, meloxicam, nabumetone, oxaprozin, piroxicam, salsalate, sulindac, and tolmetin. In one embodiment, the amount of ibuprofen is about 200 mg to about 800 mg. In one embodiment, the amount of aspirin is about 325 mg to about 1000 mg.

In one example, the NSAID is ibuprofen and the co-agent is fexofenadine. In one embodiment, the amount of ibuprofen is about 150 mg to about 900 mg, and the amount of fexofenadine is about 60 mg to about 180 mg. In one embodiment, the ibuprofen and the fexofenadine is combined in one unit dose. In one embodiment, the ibuprofen and the fexofenadine is in the form of a tablet, lozenge or chewing gum.

In one example, the NSAID is ibuprofen and the co-agent is ketotifen. In one embodiment, the amount of ibuprofen is about 1200 mg to about 1600 mg, and the amount of ketotifen is about 0.5 mg to about 4 mg, e.g., a daily dose of about 2400 mg to about 3200 mg ibuprofen and about 2 mg of ketotifen. In one embodiment, the ibuprofen and the ketotifen is combined in one unit dose. In one embodiment, the ibuprofen and the ketotifen is in the form of a tablet, lozenge or chewing gum.

In one embodiment, the pharmaceutical composition is administered daily beginning at most about 48 hours before last dose of an opioid was taken. In one embodiment, the pharmaceutical composition is administered at least beginning one day after last dose of an opioid was taken.

In one embodiment, the present invention is a pharmaceutical composition comprising a) an NSAID, and/or a salt thereof; and b) a co-agent selected from the group consisting of: fexofenadine, ketotifen, desloratadine, cetirizine salts thereof and combinations thereof. In one embodiment, the composition is in the form of an orally-dissolving tablet or lozenge. In one embodiment, the NSAID is ibuprofen and the co-agent is fexofenadine. In one embodiment, the amount of ibuprofen is about 150 mg to about 900 mg, and the amount of fexofenadine is about 25 mg to about 200 mg. In one embodiment, the NSAID is ibuprofen and the co-agent is ketotifen. In one embodiment, the amount of ibuprofen is about 1200 mg to about 1600 mg, and the amount of ketotifen is about 1 mg to about 2 mg.

### DETAILED DESCRIPTION OF THE INVENTION

In one embodiment, the present invention is directed to methods of inhibiting the adverse symptoms associated with opioid withdrawal in human subjects. The methods include the administration of particular pharmaceutical compositions.

Throughout this specification, quantities are defined by ranges, and by lower and upper boundaries of ranges. Each lower boundary can be combined with each upper boundary to define a range. The lower and upper boundaries should each be taken as a separate element.

Opioids are substances that act by binding to opioid receptors, which receptors are found principally in the central and peripheral nervous system and the gastrointestinal tract. These receptors mediate both the psychoactive and the somatic effects of opioids. Medically opioids are primarily used for pain relief, including anesthesia. Other medical uses include suppression of diarrhea and suppressing cough.

Opioids include opiates, which are alkaloid compounds naturally found in the opium poppy plant (i.e., Papaver somniferum). The psychoactive compounds found in the opium plant include opium, heroin, morphine, codeine and thebaine. Examples of synthetic, or semi-synthetic, opioids include hydrocodone (e.g., Vicodin®, Lorcet®, Lortab®, Percocet®, Percodan®); oxycodone (e.g., OxyContin®); fentanyl (e.g., Duragesic®); methadone (Dolophine®); pethidine (e.g., Demerol®); and hydromorphone (e.g., Dilaudid®).

Dependence develops in subjects with continuous use of an opioid leading to a withdrawal syndrome upon abrupt discontinuation of the opioid.

Opioid withdrawal symptoms may range from mild to severe, depending on how dependent the subject is on the opioid. Dependency can be directly tied to the length of time taking an opioid, dosage amount, which particular opioid was taken, route of administration, underlying medical conditions, presence of a mental health issue, and certain biological and environmental factors, such as family history of addiction, previous trauma, and stressful surroundings.

Withdrawal from an opioid may roughly adhere to the following timeline, although it can vary from subject to subject. Early withdrawal symptoms typically start within 6-12 hours after last dose is taken for short-acting opioids, and start within 30 hours after last dose is taken for longer-acting opioids. Early withdrawal symptoms include: lacrimation, muscle aches, agitation, insomnia, excessive yawning, anxiety, panic, rhinorrhea, sweating, tachycardia, hypertension and fever. Late withdrawal symptoms typically peak within 72 hours after last dose is taken, usually lasting about a week, and include nausea and vomiting, diarrhea, piloerection, stomach cramps, depression, and drug cravings. Other withdrawal symptoms may include, for example, mydriasis, restlessness; tremor; involuntary movements; muscle twitches; abdominal cramps; cold flashes; substantial physical and mental fatigue; dysphoric mood; drowsiness; salivation; loss of appetite; headache; dizziness; fainting; malaise; shivering; muscle/joint pain; irritability; poor concentration; confusion; flu-like symptoms; and the like.

The methods of the present invention comprise the administration of a pharmaceutical composition to a human subject, in need thereof, in an amount which is effective to inhibit the adverse symptoms associated with withdrawal from opioids. A human subject in need thereof is a human who is withdrawing from opioid use. Administration includes administration by a physician or by self-administration.

The pharmaceutical composition comprises a) at least one non-steroidal anti-inflammatory drug (“NSAID”), and b) a co-agent.

The NSAID of the present invention includes any NSAID and salts thereof. Examples of suitable NSAIDs include, but are not limited to, aspirin (i.e., acetylsalicylic acid); ibuprofen (i.e., isobutylphenylpropanoic acid); naproxen (i.e., 6-methoxy-α-methyl-2-naphthaleneacetic acid); diclofenac (i.e., 2-[(2,6-dichlorophenyl)-amino]benzene acetic acid); diflunisal (i.e., 2′,4′-difluoro-4-hydroxybiphenyl-3-carboxylic acid); etodolac (i.e., (RS)-2-(1,8-diethyl-4,9-dihydro-3H-pyrano[3,4-b]indol-1-yl)acetic acid); indomethacin (i.e., 2-{1-[(4-chlorophenyl)-carbonyl]-5-methoxy-2-methyl-1H-indol-3-yl}acetic acid); ketoprofen (i.e., 3-benzoyl-α-methyl-benzeneacetic acid); ketorolac (i.e., 2-amino-2-(hydroxymethyl)-1,3-propanediol); meloxicam (i.e., 4-hydroxy-2-methyl-N-(5-methyl-2-thiazolyl)-2H-1,2-benzothiazine-3-carboxamide-1,1-dioxide); nabumetone (i.e., 4-(6-methoxy-2-naphthyl)-2-butanone); oxaprozin (i.e., 3-(4,5-diphenyl-1,3-oxazol-2-yl)propanoic acid); piroxicam (i.e., 4-hydroxy-2-methyl-N-2-pyridinyl-2H-1,2-benzothiazine-3-carboxamide 1,1-dioxide); salsalate (i.e., 2-(2-hydroxybenzoyl)-oxybenzoic acid); sulindac (i.e., {(1Z)-5-fluoro-2-methyl-1-[4-(methylsulfinyl)-benzylidene]-1H-indene-3-yl}acetic acid); and tolmetin (i.e., [1-methyl-5-(4-methylbenzoyl)-1H-pyrrol-2-yl]acetic acid).

Suitable co-agents include desloratadine (i.e., 8-chloro-6,11-dihydro-11-(4-piperdinylidene)-5H-benzo[5.6]cyclohepta[1,2-b]pyridine); fexofenadine (i.e., (±)-4-[1 hydroxy-4-[4-(hydroxydiphenylmethyl)-1-piperidinyl]-butyl]-α, α-dimethyl benzeneacetic acid); ketotifen; cetirizine; and salts of such co-agents.

The NSAIDs and co-agents include all pharmaceutically acceptable versions of the NSAIDs and co-agents, including, for example, stereoisomers and/or any mixtures thereof, all pharmaceutically acceptable zwitterions and/or any mixtures thereof, all pharmaceutically acceptable polymorphic forms and/or any mixtures thereof, and all pharmaceutically acceptable complexes (including solvates) and/or any mixtures thereof.

Salts include all salts of NSAIDs and of co-agents which are pharmaceutically acceptable (i.e., non-toxic at therapeutically effective doses). And, salts include their racemates, enantiomers, or any mixtures thereof.

Particularly suitable salts of the NSAIDs comprise alkali-metal salts (e.g., sodium and/or potassium salts), alkaline earth metal salts (e.g., magnesium and/or calcium salts), aluminum salts, ammonium salts, salts of suitable organic bases (e.g., salts of alkylamines and/or -methyl-D-glutamine), salts of amino acids (e.g., salts of arginine and/or lysine). The NSAID salts also include all enantiomeric salts formed with pharmaceutically acceptable chiral acids and/or bases and/or any mixtures of enantiomers of such salts (e.g., (+) tartrates, (−) tartrates and/or any mixtures thereof including racemic mixtures). For example, a typical salt of an NSAID is naproxen sodium.

Examples of suitable salts of the co-agents include ketotifen fumarate, fexofenadine hydrochloride and cetirizine hydrochloride.

In one embodiment, the pharmaceutical composition is administered to the human subject, in need thereof, prior to taking the last dose of an opioid. For example, administration is at most about 48 hours before the last dose of an opioid, or at most about 24 hours before the last dose of an opioid, or at most about twelve hours before the last dose of an opioid, or at most about six hours before the last dose of an opioid, or at most about three hours before the last dose of an opioid, or at most about one hour before the last dose of an opioid, or right before the last dose of an opioid.

In another embodiment, the pharmaceutical composition is administered to the human subject, in need thereof, right after or about 6-12 hours after the last dose taken of a short-acting opioid, and about 30 hours after the last dose taken of a long-acting opioid. For example, administration is at least about 6 hours after the last dose of an opioid, and at most about 7 hours after the last dose of an opioid, or at most about 12 hours after the last dose of an opioid, or at most about one day after the last dose of an opioid, or at most about two days after the last dose of an opioid, or at most about three days after the last dose of an opioid, or at most about five days after the last dose of an opioid, or at most about six days after the last dose of an opioid.

In one embodiment, the pharmaceutical composition is administered during methadone detoxification therapy. Such therapy can either be done relatively rapidly in less than a month or gradually over as long as six months.

Adverse symptoms associated with opioid withdrawal can last for about two to eight weeks. During such period, the pharmaceutical composition can be administered on a substantially daily basis. Daily NSAID use has been associated with adverse gastrointestinal effects (e.g., upset stomach, ulcers). However, when the NSAIDs of the present invention are taken in combination with the co-agents, adverse gastrointestinal effects are surprisingly slight or absent. Thus, it has unexpectedly been found that the components of the compositions of the present invention have a synergistic effect when inhibiting the adverse symptoms associated with the withdrawal from opioids.

In the present specification, the term “inhibit” includes “reduce” and/or “prevent” and/or “shorten duration.” That is, the method of the present invention is considered to be effective if it causes one or more of: a reduction/prevention of any adverse symptom associated with withdrawal from opioids and/or shortening of the duration of an episode of any such adverse withdrawal symptom.

Inhibition of adverse withdrawal symptoms can be assessed by comparing the magnitude and/or duration of at least one symptom in a subject at two different occasions, that is, i) when administered the pharmaceutical composition during a withdrawal period; and ii) when not administered the pharmaceutical composition during a withdrawal period. An assessment is made as to the severity of withdrawal symptoms at the different occasions.

Inhibition of adverse withdrawal symptoms can also be assessed by comparing the magnitude and/or duration of at least one symptom in different subjects withdrawing from the same opioid, some of whom are administered the pharmaceutical composition during a withdrawal period and some whom are not administered the pharmaceutical composition during a withdrawal period. An assessment is made as to the severity of withdrawal symptoms between the different subjects.

Typically, adverse withdrawal symptoms are inhibited by at least about 10%, at least about 20%, at least about 30%, at least about 40%, at least about 50%, at least about 60%, at least about 70%, at least about 80%, at least about 90%, or about 100%.

The actual preferred amounts of a pharmaceutical composition in a specified case will vary according to the particular composition formulated, the mode of application, the particular sites of application, and the subject being treated (e.g., age, gender, size, tolerance to drug, etc.).

Examples of typical daily amounts of NSAIDs to be administered in the methods of the present invention follows. The daily amounts can be administered in one dose, or in multiple doses, typically, two doses.

Naproxen from about 110 mg to about 1500 mg: Examples of other lower boundaries of this range include about 150 mg, about 220 mg, about 275 mg, about 320 mg and about 420 mg. Examples of other upper boundaries of this range include about 580 mg, about 680 mg, about 780 mg, about 880 mg and about 950 mg.

Ibuprofen from about 100 mg to about 3200 mg: Examples of other lower boundaries of this range include about 200 mg, about 400 mg, about 600 mg, about 700 mg, about 800 mg, about 950 mg and about 1000 mg. Examples of other upper boundaries of this range include about 1200 mg, about 1500 mg, about 1600 mg, about 2000 mg, about 2500 mg and about 3000 mg.

Aspirin from about 250 mg to about 4000 mg: Examples of other lower boundaries of this range include about 325 mg, about 450 mg, about 550 mg, about 700 mg, about 1000 mg, about 1500 mg, and about 1800 mg. Examples of other upper boundaries of this range include about 2000 mg, about 2500 mg, about 3000 mg, about 3500 mg, and about 3800 mg.

Examples of typical daily amounts of the co-agent to be administered in the methods of the present invention follows. The daily amounts can be administered in one dose, or in multiple doses, typically, two doses.

Fexofenadine from about 25 mg to about 200 mg: Examples of other lower boundaries of this range include about 60 mg, about 70 mg, about 80 mg and about 90 mg. Examples of other upper boundaries of this range include about 100 mg, about 120 mg, about 150 mg and about 180 mg. Ketotifen from about 0.5 mg to about 3 mg, or about 0.5 mg to about 4 mg, or about 0.5 mg to about 4 mg: Examples of other lower boundaries of this range include about 1 mg, about 1.5 mg and about 1.8 mg. Examples of other upper boundaries of this range include about 2 mg, about 2.5 mg and about 2.8 mg, and about 3.5 mg. Desloratidine from about 2 mg to about 40 mg: Examples of other lower boundaries of this range include about 5 mg, about 6 mg and about 7 mg. Examples of other upper boundaries of this range include about 8 mg, about 9 mg and about 10 mg. Cetirizine from about 2 mg to about 10 mg: Examples of other lower boundaries of this range include about 5 mg, about 6 mg and about 7 mg. Examples of other upper boundaries of this range include about 8 mg, about 9 mg and about 10 mg.

In one embodiment of the invention, a pharmaceutical composition comprises about 800 mg ibuprofen and about 60 mg fexofenadine. For example, the pharmaceutical composition can be administered every twelve hours beginning before the last dose of an opioid is taken, preferably by delayed release administration.

In one embodiment of the invention, a pharmaceutical composition comprises about 1200 mg to about 1600 mg ibuprofen and about 1 mg ketotifen, administered to result in a daily dose of about 2400 mg to about 3200 mg ibuprofen and about 2 mg ketotifen. For example, the pharmaceutical composition can be administered every twelve hours beginning before the last dose of an opioid is taken, preferably by controlled release administration.

The pharmaceutical composition can be administered by methods known in the art. For example, the pharmaceutical composition can be administered systemically. For the purposes of this specification, “systemic administration” means administration to a human by a method that causes the compositions to be absorbed into the bloodstream.

In one embodiment, the pharmaceutical compositions are administered orally by any method known in the art. For example, the compositions can be administered in the form of tablets, including, e.g., orally-dissolvable tablets, chewable tablets; capsules; lozenges; pills (e.g., pastilles, dragees); troches; elixirs; suspensions; syrups; wafers; chewing gum; strips; films (e.g., orally-dissolving thin films); soluble powders; effervescent compositions; and the like.

The NSAID (and/or salt thereof) and the co-agent can be supplied in combination as one unit dose, or can be supplied individually, e.g., supplied in a package with a unit dose of NSAID and a unit dose of the co-agent.

Additionally, the pharmaceutical compositions can be administered enterally or parenterally, e.g., intravenously; intramuscularly; subcutaneously, as injectable solutions or suspensions; intraperitoneally; sublingually; or rectally (e.g., by suppositories). Administration can also be intranasally, in the form of, for example, an intranasal spray; or transdermally, in the form of, for example, a patch.

The pharmaceutical composition compounds of the invention can be formulated per se in pharmaceutical preparations, optionally, with a suitable pharmaceutical carrier (vehicle) or excipient, as understood by practitioners in the art. These preparations can be made according to conventional chemical methods.

In the case of tablets for oral use, carriers commonly used include lactose and corn starch, and lubricating agents such as magnesium stearate are commonly added. For oral administration in capsule form, useful carriers include lactose and corn starch. Further examples of carriers and excipients include milk, sugar, certain types of clay, gelatin, stearic acid or salts thereof, calcium stearate, talc, vegetable fats or oils, gums and glycols.

When aqueous suspensions are used for oral administration, emulsifying and/or suspending agents are commonly added. In addition, sweetening and/or flavoring agents may be added to the oral compositions.

For intramuscular, intraperitoneal, subcutaneous and intravenous use, sterile solutions of the pharmaceutically compositions can be employed, and the pH of the solutions can be suitably adjusted and buffered. For intravenous use, the total concentration of the solute(s) can be controlled in order to render the preparation isotonic.

A preferred embodiment of the invention is an orally dissolving tablet comprising an NSAID and a coagent with or without a taste masking ingredient, diluents, etc. Such tablet can be administered without water onto the tongue leading to immediate dissolution and is absorbed gastrointestinally or buccally. Orally dissolving tablets can be formulated by a number of techniques including compression and lyophilization, as would be known to a skilled artisan.

Another preferred embodiment of the invention is a lozenge or troche comprising an NSAID and a coagent with or without a taste masking ingredient, diluents, etc. Such lozenge/troche can be administered without water, and can slowly dissolve in the mouth, or can be swallowed or chewed. Such lozenges/troches can be formulated by compression, as would be known to a skilled artisan.

The pharmaceutical compositions of the present invention can further comprise one or more pharmaceutically acceptable additional ingredient(s) such as alum, stabilizers, buffers, coloring agents, flavoring agents, and the like. In some embodiments, orally administered pharmaceutical compositions can contain breathe neutralizers, e.g., peppermint or menthol scents.

The pharmaceutical composition may be administered by controlled release. Controlled release administration is a method of drug delivery to achieve a certain level of the drug over a particular period of time. The level typically is measured by plasma concentration. Methods for controlled release of drugs are well known in the art.

The pharmaceutical compositions can be formulated for controlled release. For example, in one embodiment, the composition can be a capsule containing beadlets, wherein some of the beadlets dissolve instantaneously and some of the beadlets dissolve at delayed times due to different types of beadlet coatings. For example, a controlled release composition can be administered twice a day, twelve hours apart.

In one embodiment, the pharmaceutical composition comprises an active ingredient, wherein the active ingredient consists of: a) NSAID, and/or salt thereof, and b) a co-agent selected from the group consisting of: fexofenadine, ketotifen, desloratadine, cetirizine, salts thereof and combinations thereof.

In one embodiment, the pharmaceutical composition consists of: a) NSAID, and/or salt thereof, b) a co-agent selected from the group consisting of: fexofenadine, ketotifen, desloratadine, cetirizine, salts thereof, and combinations thereof; and c) at least one carrier and/or excipient.

In one embodiment, the pharmaceutical composition consists essentially of the active ingredients of: a) NSAID and/or salt thereof, and b) a co-agent selected from the group consisting of: fexofenadine, ketotifen, desloratadine, cetirizine, salts thereof and combinations thereof. That is, any other ingredients that may materially affect the basic and novel characteristics of the active ingredients of the invention are specifically excluded from the composition. Any ingredient which can potentially cause an undesirable effect/side effect, including, for example, an allergic response, may materially affect the basic and novel characteristics of the active ingredients of the invention.

The following are some examples of components which may materially affect the basic and novel characteristics of the active ingredients of the pharmaceutical compositions and may be excluded from certain embodiments of the present invention: cyclooxygenase-2-selective inhibitors (i.e., COX-2-selective inhibitors) or prodrugs thereof; sedating antihistamines (e.g., phenyltoloxamine (e.g., phenyltoloxamine citrate), doxylamine (e.g., doxylamine succinate)); antiemetic antihistamines (e.g., dimenhydrinate (Dramamine®), clizines (e.g., cyclizine, meclizine), diphenhydramine (Benadryl®), promethazine (Pentazine®, Phenergan®, Promacot®), and hydroxyzine (Vistaril®)); decongestants; flunixin meglumine (i.e., banamine); 5-HT3 receptor antagonists; cough suppressants (e.g., guaifenesin, dextromethorphan); H₂ antihistamines; and corticosteroids.

The aforementioned ingredients may materially change the characteristics of the present pharmaceutical composition due to unwanted effects and/or potential allergic responses.

Examples of unwanted potential effects of COX-2-selective inhibitors, or prodrugs thereof, include an increased risk in the incidence of myocardial infarctions. COX-2-selective inhibitors are compounds which selectively inhibit cyclooxygenase-2 over cyclooxygenase-1, and also include pharmaceutically acceptable salts of such compounds, and prodrugs of such compounds. A COX-2 selective inhibitor is any inhibitor for which the ratio of COX-1 IC₅₀ to COX-2 IC₅₀ is greater than 1. Examples of unwanted potential effects of sedating antihistamines, decongestants, and diphenhydramine include sleepiness, fatigue, dizziness, headache, dry mouth, difficulty urinating or an enlarged prostate and allergic reactions. Examples of unwanted potential effects of flunixin meglumine include ataxia, incoordination, hyperventilation, hysteria and muscle weakness. Examples of unwanted potential effects of 5-HT3 receptor antagonists include constipation, diarrhea, headache, dizziness and arrhythmias. Examples of unwanted potential effects of guaifenesin include diarrhea, dizziness, headache, hives, nausea or vomiting, skin rash and stomach pain. Examples of unwanted potential effects of dextromethorphan include confusion, constipation, dizziness, drowsiness, headache, nausea or vomiting and stomach pain. Examples of unwanted potential effects of H₂ antihistamines include abdominal pain, bleeding or crusting sores on lip, dizziness, fainting, fever and chills. Examples of unwanted potential effects of corticosteroids include fluid retention, edema, weight gain, high blood pressure, headache and muscle weakness.

Thus, while there have been described what are presently believed to be the preferred embodiments of the present invention, other and further embodiments, modifications, and improvements will be known to those skilled in the art, and it is intended to include all such further embodiments, modifications, and improvements as come within the true scope of the claims as set forth below.

1. A method of inhibiting symptoms associated with opioid withdrawal in a human subject in need thereof, comprising:
administering an effective amount of a pharmaceutical composition to the subject during an opioid withdrawal period, wherein the pharmaceutical composition comprises:
a) a non-steroidal anti-inflammatory drug (NSAID); and
b) a co-agent selected from the group consisting of: fexofenadine, ketotifen, desloratadine and cetirizine and combinations thereof;
wherein the symptoms associated with opioid withdrawal are inhibited in the human subject.

2. The method of claim 1 wherein the NSAID is selected from the group consisting of: aspirin, ibuprofen, naproxen, diclofenac, diflunisal, etodolac, indomethacin, ketoprofen, ketorolac, meloxicam, nabumetone, oxaprozin, piroxicam, salsalate, sulindac, and tolmetin.

3. The method of claim 1 wherein the NSAID is ibuprofen and the co-agent is ketotifen.

4. The method of claim 1 wherein the amount of ibuprofen is about 1200 mg to about 1600 mg, and the amount of ketotifen is about 0.5 mg to about 3 mg.

5. The method of claim 1 wherein the daily dose of ibuprofen is about 2400 mg to about 3200 mg, and the daily dose of ketotifen is about 2 mg.

6. The method of claim 4 wherein the ibuprofen and the ketotifen is combined in one unit dose.

7. The method of claim 6 wherein the ibuprofen and the ketotifen is in the form of a tablet, lozenge or chewing gum.

8. The method of claim 1 wherein the pharmaceutical composition is administered daily beginning at most about 48 hours before last dose of an opioid was taken.

9. The method of claim 1 wherein the pharmaceutical composition is administered at least beginning one day after last dose of an opioid was taken.

10. The method of claim 1 wherein the symptoms include rhinorrhea, coughing, fever, and/or muscle/joint pain.

11. The method of claim 1 wherein the amount of ibuprofen is about 200 mg to about 800 mg.

12. The method of claim 1 wherein the amount of aspirin is about 325 mg to about 1000 mg.

13. The method of claim 1 wherein the amount of fexofenadine is about 60 mg to about 180 mg.

14. The method of claim 1 wherein the amount of desloratidine is about 5 mg to about 10 mg.

15. A method of inhibiting the symptoms associated with withdrawal from an opioid in a human subject in need thereof, comprising:
administering to the subject an effective amount of a pharmaceutical composition during an opioid withdrawal period, wherein the pharmaceutical composition consists essentially of:
a) about 1200 mg to about 1600 mg of ibuprofen, and
b) about 0.5 mg to about 3 mg ketotifen,
wherein the symptoms associated with opioid withdrawal are inhibited in the human subject.

16. A pharmaceutical composition comprising a) an NSAID, and/or a salt thereof; and b) a co-agent selected from the group consisting of: fexofenadine, ketotifen, desloratadine, cetirizine salts thereof and combinations thereof.

17. The pharmaceutical composition of claim 16 wherein the composition is in the form of an orally-dissolving tablet or lozenge.

18. The pharmaceutical composition of claim 16 wherein the NSAID is selected from the group consisting of: aspirin, ibuprofen, naproxen, diclofenac, diflunisal, etodolac, indomethacin, ketoprofen, ketorolac, meloxicam, nabumetone, oxaprozin, piroxicam, salsalate, sulindac, and tolmetin.

19. The pharmaceutical composition of claim 16 wherein the NSAID is ibuprofen and the co-agent is ketotifen.

20. The pharmaceutical composition of claim 16 wherein the amount of ibuprofen is about 1200 mg to about 1600 mg, and the amount of ketotifen is about 0.5 mg to about 3 mg.
